(19) United States
(12) Patent Application Publication
(10) Pub. No.: US 2021/0253511 A1
(43) Pub. Date: Aug. 19, 2021

(54) COMPOUNDS AND USES FOR THE TREATMENT AND PREVENTION OF DISEASES AND CONDITIONS ASSOCIATE WITH OR AGGREVATED BY IMPARED MITOPHAGY

(71) Applicant: Yissum Research Development Company of The Hebrew University of Jerusalem Ltd., Jerusalem (IL)

(72) Inventors: Shmuel BEN-SASSON, Jerusalem (IL); Einav GROSS, Jerusalem (IL)

(73) Assignee: Yissum Research Development Company of The Hebrew University of Jerusalem Ltd., Jerusalem (IL)

(21) Appl. No.: 16/980,401

(22) PCT Filed: Mar. 13, 2019
(86) PCT No.: PCT/IL2019/050278

§ 371 Date: Sep. 13, 2020

Related U.S. Application Data
(60) Provisional application No. 62/642,402, filed on Mar. 13, 2018.

(51) Int. Cl. C07C 207/02; C07C 279/04; C07C 243/14; C07C 211/01; C07D 233/54; C07D 213/02; C07D 295/00

(52) U.S. Cl. CPC C07C 207/02; C07C 279/04; C07C 243/14; C07D 295/00; C07D 233/54; C07D 213/02; C07C 211/01

(57) ABSTRACT

The present invention provides compounds and methods for the treatment and prevention of diseases and conditions associate with or aggravated by impaired mitophagy.

### BACKGROUND OF THE INVENTION

Mitochondria (MT) are double-membrane-bound organelles found in most eukaryotic organisms. They are essential for chemical energy production, in the form of ATP, in all aerobic organisms, including humans. Moreover, mitochondria are essential for many other metabolic processes including the synthesis of amino acids, lipids, heme, steroid hormones, and are the source for reactive oxygen species (ROS).

ROS present cells with a double-edged sword. On the one hand, they play a crucial role in many cellular and physiological processes including the innate immune response and the degradation and recycling of the cellular milieu in a process called autophagy. On the other hand, ROS interact with metals to produce toxic oxygen (O₂) radicals that can damage all biological molecules and thus interfere with mitochondria function and cause cell injury and death. Mitochondria themselves are generating ROS, as part of their physiological activity, and are especially vulnerable to ROS-induced damage. Therefore, to maintain healthy mitochondria there is a constant need to generate new mitochondria components (mitochondrial biogenesis) while removing the damaged one (mitophagy=mitochondrial-autophagy).

The proper functioning of this intracellular “quality-control” mechanism of mitophagy is especially important in tissues where no renewal by cell-division is taking place. Cells of nonrenewable tissues include neurons, skeletal muscle and heart muscle cells, insulin-producing beta-cells of the endocrine pancreas, cells of the retinal pigment epithelium and more. Indeed, degenerative diseases associated with aging belong mainly to such nonrenewable tissue including dementia, Alzheimer's and Parkinson's diseases, sarcopenia (=skeletal muscle atrophy), congestive heart failure, type 2 diabetes, age-related macular degeneration and more.

While the biogenesis of mitochondria does not generally decline with age (and may even increase), mitophagy is profoundly decreased. Therefore, the accumulation of damaged mitochondria is thought to underlie the decline in organ function and health span. The current consensus is that impaired mitophagy plays a pivotal role in the development of these degenerative diseases associated with aging (Markaki M. et al. Int Rev Cell Mol Biol (2018) 340: 169-208).

It was shown that subjects with Parkinson's disease (PD) have compromised mitophagy processes (Lee S H et al. (2016) EMBO Mol Med 8:779-85; Gao F. et al. Frot Neurol (2017) 8:527). Indeed, mitochondrial dysfunction appears to be a key factor in the pathophysiology of both familial and sporadic PD as well as in cases of toxin-induced Parkinsonism (Rayn B J. et al. Trends Biochem Sci (2015) 40:200-10).

The toxin 1-methyl 4-phenyl 1,2,3,6-tetrahydropyridine (MPTP) induces Parkinsonian syndrome in people (Langston W J. et al. Science (1983) 219: 979-980). Since the chemical structures MPTP and the pesticide N,N′-dimethyl-4,4′-bipyridinium dichloride (paraquat) are similar, paraquat is widely used in animal model of PD (Miller G W. Toxicol Sci (2007) 100: 1-2). Moreover, paraquat (PQ) is a robust inducer of oxidative stress in cells (Halliwell B., Gutteridge J. in Free Radicals in Biology and Medicine, Clarendon Press, Oxford, 2006.). Therefore, induction of mitophagy should increase the resistance to PQ-induced oxidative injury. In agreement with the above, it was shown that compromising C. elegans mitophagy make this organism more vulnerable to PQ toxicity (Luz A L et al. Toxicology (2017) 387:81-94).

Thus, it is well established that the protection of cells and organisms against paraquat-induced damage is a hallmark of mitophagy augmentation (Dagda R A et al. Int. J. Mol. Sci. 14: 22163-89 (2013)).

There is a need for a medicaments and methods capable of treating and/or protecting the human body from the damages of impaired mitophagy/mitochondrial-autophagy, including any conditions, diseases, disorders and symptoms associated therewith and also including conditions and diseases associated with cell degeneration, in particular in cells of non-regenerative tissues.

### SUMMARY OF THE INVENTION

The present invention provides a compound having a general formula (I), for use in the treatment, slowing down the progression or prevention of a condition, disease, disorder or symptom associated with cell degeneration;

R₁-L-R₂ (I)
wherein R₁ and R₂ are each independently selected from —C(═NR₃)NR₄R₅ (amidine), —NR₆R₇ (amine), —N⁺R₈R₉R₁₀, —NR₁₁C(═N)NR₁₂R₁₃ (guanidine), —NR₁₄C(═N)—NR₁₅—C(═N)—NR₁₆R₁₇, —NR₁₈NR₁₉R₂₀ (hydrazine), ═N—R₂₁ (imine), —ONR₂₂R₂₃ (aminooxy),

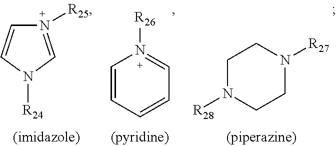

wherein each of R₃— R₂₈ is independently selected from H, straight or branched C₁-C₁₂ alkyl, straight or branched C₂-C₁₂ alkenyl, straight or branched C₂-C₁₂ alkynyl, phenyl, —OH, halogen and any combinations thereof;
L is selected from straight or branched C₆-C₁₂ alkylene, straight or branched C₈-C₁₂ alkenylene, straight or branched C₆-C₁₂ alkynylene;
each defined L optionally interrupted by at least one of C₄-C₈ cycloalkylene, C₄-C₈ cycloalkenylene, C₄-C₈ cycloalkynylene, arylene, heteroarylene, heteroatom and any combinations thereof;
each defined L optionally substituted with at least one of halogen and any combinations thereof.

It should be understood that the term “interrupted by” as used herein refers to the option wherein at least one moiety as listed herein above is connected between any two carbon atoms of L, thus said at least one moiety has two open valencies. Furthermore, the term “substituted with” should be understood to relate to the option of substituting at least one hydrogen atom of L with at least one moiety as listed herein above, thus said at least one moiety has one open valency.

In some embodiments, L is straight or branched C₆-C₁₂ alkylene. In some embodiments, L is straight or branched C₆-C₈ alkylene. In some embodiments, L is straight or branched C₈-C₁₂ alkylene. In some embodiments, L is straight or branched C₁₀-C₁₂ alkylene. In some embodiments, L is straight or branched C₆ alkylene. In some embodiments, L is straight or branched C₇ alkylene.

In some embodiments, L is straight or branched C₈ alkylene. In some embodiments, L is straight or branched C₉ alkylene. In some embodiments, L is straight or branched C₁₀ alkylene. In some embodiments, L is straight or branched C₁₁ alkylene. In some embodiments, L is straight or branched C₁₂ alkylene.

In other embodiments, L is interrupted by at least one of C₄-C₈ cycloalkylene, C₄-C₈ cycloalkenylene, C₄-C₈ cycloalkynylene, aryl, heteroaryl, heteroatom and any combinations thereof. In further embodiments, L is interrupted by at least one C₄-C₈ cycloalkylene. In other embodiments, L is interrupted by at least one C₄-C₈ cycloalkenylene. In further embodiments, L is interrupted by at least one C₄-C₈ cycloalkynylene. In other embodiments, L is interrupted by at least one aryl selected from phenylene or biphenylene. In further embodiments, L is interrupted by at least one heteroarylene. In yet other embodiments, L is interrupted by at least one heteroatom selected from N, O, S.

In some embodiments, L is substituted with at least one of halogen selected from F, Br, Cl, I and any combinations thereof. In some embodiments, L is substituted with at least one F. In some embodiments, L is substituted with at least one Br. In some embodiments, L is substituted with at least one Cl. In some embodiments, L is substituted with at least one I.

In some embodiments, R₁ and R₂ are identical. In other embodiments, R₁ and R₂ are different.

In some embodiments, R₁ and R₂ are each —C(═NR₃)NR₄R₅ (amidine), wherein R₃-R₅ are as defined herein above and may be the same or different for each R₁ or R₂.

In other embodiments, R₁ and R₂ are each selected from —NR₆R₇ (amine) and —N⁺R₈R₉R₁₀ wherein R₆-R₁₀ are as defined herein above and may be the same or different for each R₁ or R₂

In further embodiments, R₁ and R₂ are each selected from —NR₁₁C(═N)NR₁₂R₁₃ (guanidine) and —NR₁₄C(═N)—NR₁₅—C(═N)—NR₁₆R₁₇ wherein R₁₁-R₁₇ are as defined herein above and may be the same or different for each R₁ or R₂

In other embodiments. R₁ and R₂ are each —NR₁₈NR₁₉R₂₀ (hydrazine), wherein R₁₈-R₂₀ are as defined herein above and may be the same or different for each R₁ or R₂

In other embodiments, R₁ and R₂ are each ═N—R₂₁ (imine), wherein R₂₁ is as defined herein above and may be the same or different for each R₁ or R₂

In other embodiments, R₁ and R₂ are each —ONR₂₂R₂₃ (aminooxy), wherein R₂₂-R₂₃ are as defined herein above and may be the same or different for each R₁ or R₂

In other embodiments, R₁ and R₂ are each

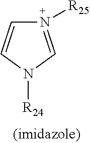

wherein R₂₄-R₂₅ are as defined herein above and may be the same or different for each R₁ or R₂

In further embodiments, R₁ and R₂ are each

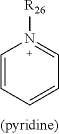

wherein R₂₆ is as defined herein above and may be the same or different for each R₁ or R₂.

In further embodiments, R₁ and R₂ are each

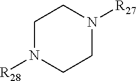

wherein R₂₇ and R₂₈ are as defined herein above and may be the same or different for each R₁ or R₂.

The invention further provides a compound having a general formula (I);

R₁-L-R₂ (I)
wherein R₁ and R₂ are each independently selected from —C(═NR₃)NR₄R₅ (amidine), —NR₆R₇ (amine), —N⁺R₈R₉R₁₀, —NR₁₁C(═N)NR₁₂R₁₃ (guanidine), —NR₁₄C(═N)—NR₁₅—C(═N)—NR₁₆R₁₇, —NR₁₈NR₁₉R₂₀ (hydrazine), ═N—R₂₁ (imine), —ONR₂₂R₂₃ (aminooxy),

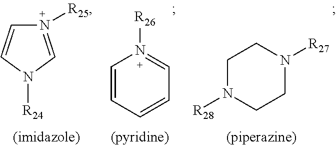

wherein each of R₃-R₂₈ is independently selected from H, straight or branched C₁-C₁₂ alkyl, straight or branched C₂-C₁₂ alkenyl, straight or branched C₂-C₁₂ alkynyl, phenyl, —OH, halogen and any combinations thereof;
L is selected from straight or branched C₆-C₁₂ alkylene, straight or branched C₆-C₁₂ alkenylene, straight or branched C₆-C₁₂ alkynylene;
each defined L interrupted by at least one of C₄-C₈ cycloalkylene, C₄-C₈ cycloalkenylene, C₄-C₈ cycloalkynylene, aryl, heteroaryl, heteroatom and any combinations thereof;
each defined L optionally substituted with at least one of halogen and any combinations thereof.

In some embodiments, a compound of the invention is selected from 1,8-diaminooxy-octane; 1-aminooxy, 9-amino-nonane; 1-aminooxy, 8-guanido-octane and 1,4-phenyl-bis-butylamine. In other embodiments, a compound of the invention is 1,4-phenyl-bis-butylamine.

In another aspect the invention provides a composition comprising at least one compound as defined herein above.

In further aspect, the invention provides a compound as defined herein above, for use it the treatment of a condition or a disease associated with cell degeneration.

When referring to “treatment of a disease, disorder, symptom, which is caused by, associated with, or aggravated by impaired mitophagy” it should be understood to encompass the management and care of a patient for the purpose of combating a disease, disorder, condition or symptom and includes the slowing the progression or delaying of the progression of the disease, disorder, condition or symptom, the alleviation or relief of symptoms and complications, and/or the cure or elimination of the disease, disorder or condition. Said condition, disease, disorder or symptom are defined to be associated with directly or indirectly, caused by directly or indirectly or directly or indirectly aggravated by impaired mitophagy process, i.e. the cellular process of removing damaged mitochondria is biologically inefficient, reduced and insufficient for the purposes of maintaining a healthy viable cell. In some embodiments, said mitophagy process is a process in cells of non-regenerative tissues.

When referring to “prevention of a disease, disorder, symptom, which is caused by, associated with, or aggravated by impaired mitophagy” it should be understood to encompass to substantially stopping the occurrence or progression of a disease, disorder, condition or symptom. Said condition, disease, disorder or symptom are defined to be associated with directly or indirectly, caused by directly or indirectly or directly or indirectly aggravated by impaired mitophagy process, i.e. the cellular process of removing damaged mitochondria is biologically inefficient, reduced and insufficient for the purposes of maintaining a healthy viable cell. In some embodiments, said mitophagy process is a process in cells of non-regenerative tissues.

When relating to the use of compounds of the invention in the “treatment of a condition, disease, disorder or symptom associated with cell degeneration”, it should be understood to relate to the management and care of a patient for the purpose of combating a disease, disorder, condition or symptom and includes the prevention or delaying of the progression of the disease, disorder, condition or symptom, the alleviation or relief of symptoms and complications, and/or the cure or elimination of the disease, disorder or condition. Said condition, disease, disorder or symptom are defined to be associated with, caused by, or aggravated by the process of inexorable slide into no functionality of cells caused by stochastic degradation of its parts, in some embodiments the mitochondria. In further embodiments, the invention is directed to the treatment of conditions, disorders, diseases or symptoms associated with cell degeneration of non-regenerative tissues. Such “non-regenerative tissue” include tissues that do not spontaneously regenerate such as neurons (central and peripheral nervous system), cardiomyocytes (heart muscle cells), skeletal-muscle cells, insulin-producing cells (beta-cells of the endocrine pancreas), and retinal pigment epithelium.

In further aspect, the invention provides a compound as defined herein above, for use it the slowing the progression of or preventing a condition or a disease associated with cell degeneration.

When referring to “slowing the progression” it should be understood to relate to delaying of the progression of the disease, disorder, condition or symptom, associated with, caused by, or aggravated by cell degeneration. In some embodiments, the invention is directed to the treatment of conditions, disorders, diseases or symptoms associated with cell degeneration of non-regenerative tissue.

When referring to “preventing” it should be understood to relate to substantially stopping the occurrence or progression of the disease, disorder, condition or symptom, associated with, caused by, or aggravated by cell degeneration. In some embodiments, the invention is directed to the treatment of conditions, disorders, diseases or symptoms associated with cell degeneration of non-regenerative tissue.

In some embodiments, said condition or a disease associated with cell degeneration is a neurodegenerative disease, disorder and condition associated therewith.

In other embodiments, said condition or a disease associated with cell degeneration is an age-related disease, disorder and condition associated therewith.

In further embodiments, said condition or a disease associated with cell degeneration is selected from Parkinson's disease, Alzheimer's disease, dementia, congestive heart failure, sarcopenia, type 2 diabetes, age-related macular degeneration (AMD), atherosclerosis, cardiovascular diseases, cancer, liver diseases, pancreatic diseases, ocular diseases, arthritis, cataracts, osteoporosis, hypertension, and any combinations thereof.

The invention further provides a compound as defined herein above and below for use in a method of maintaining the vitality of non-regenerating tissue in a subject, said method comprising administering to said subject an effective dose of a compound as defined herein above and below.

When referring to “maintaining the vitality of non-regenerating tissue” it should be understood to relate to keeping the vital state of a non-regenerating tissue by slowing down the progression or preventing said tissue cell degeneration. Upon maintaining the vitality of non-regenerative tissue, the lifespan of a subject treated with a compound of the invention can be prolonged.

The invention further provides a method of maintaining the vitality of non-regenerating tissue in a subject, said method comprising administering to said subject an effective dose of a compound as defined herein above and below.

The invention further provides a method for the treatment of a condition or a disease associated with cell degeneration in a subject, said method comprising administering to said subject an effective dose of a compound as defined herein above and below.

The invention further provides a method for slowing the progression of or preventing a condition or a disease associated with cell degeneration in a subject, said method comprising administering to said subject an effective dose of a compound as defined herein above and below.

In further aspect, the invention provides a compound as defined herein above, for use in facilitating mitophagy. When referring to the facilitation of mitophagy it should be understood to encompass the promotion of, enhancement of, enablement of the process of mitophagy in cells, thereby prolonging the viability of said cells. In some embodiments, said cells are of non-regenerative tissue.

The term “straight or branched C₁-C₁₂ alkyl” should be understood to encompass any straight or branched saturated hydrocarbon chain having 1, 2, 3, 4, 5, 6, 7, 8, 9, 10, 11 or 12 carbon atoms, wherein only sigma bonds connect between the atoms of the chain, and wherein one hydrogen atom is removed from any carbon atom of the chain.

The term “straight or branched C₂-C₁₂ alkenyl” should be understood to encompass any straight or branched unsaturated hydrocarbon chain having 2, 3, 4, 5, 6, 7, 8, 9, 10, 11 or 12 carbon atoms, wherein at least one double bond connects two carbon atoms at any point of the hydrocarbon chain, and wherein one hydrogen atom is removed from any carbon atom of the chain.

The term “straight or branched C₂-C₁₂ alkynyl” should be understood to encompass any straight or branched unsaturated hydrocarbon chain having 2, 3, 4, 5, 6, 7, 8, 9, 10, 11 or 12 carbon atoms, wherein at least one triple bond connects two carbon atoms at any point of the hydrocarbon chain, and wherein one hydrogen atom is removed from any carbon atom of the chain.

The term “straight or branched C₆-C₁₂ alkylene” should be understood to encompass any straight or branched saturated hydrocarbon chain having 6, 7, 8, 9, 10, 11 or 12 carbon atoms, wherein only sigma bonds connect between the atoms of the chain, and wherein two hydrogen atoms are removed from any two carbon atoms of the chain.

The term “straight or branched C₆-C₁₂ alkenylene” should be understood to encompass any straight or branched unsaturated hydrocarbon chain having 6, 7, 8, 9, 10, 11 or 12 carbon atoms, wherein at least one double bond connects two carbon atoms at any point of the hydrocarbon chain, and wherein two hydrogen atoms are removed from any two carbon atoms of the chain.

The term “straight or branched C₆-C₁₂ alkynylene” should be understood to encompass any straight or branched unsaturated hydrocarbon chain having 6, 7, 8, 9, 10, 11 or 12 carbon atoms, wherein at least one triple bond connects two carbon atoms at any point of the hydrocarbon chain, and wherein two hydrogen atoms are removed from any two carbon atoms of the chain.

The term “C₄-C₈ cycloalkylene” should be understood to encompass any saturated cyclic hydrocarbon ring having 4, 5, 6, 7, 8, carbon atoms, wherein only sigma bonds connect between the atoms of the ring, and wherein two hydrogen atoms are removed from any carbon atoms of the ring.

The term “C₄-C₈ cycloalkenylene” should be understood to encompass any cyclic unsaturated hydrocarbon ring having 4, 5, 6, 7, 8 carbon atoms, wherein at least one double bond connects two carbon atoms at any point of the hydrocarbon ring, and wherein two hydrogen atoms are removed from any two carbon atoms of the ring.

The term “C₄-C₈ cycloalkynylene” should be understood to encompass any cyclic unsaturated hydrocarbon ring having 4, 5, 6, 7, 8 carbon atoms, wherein at least one triple bond connects two carbon atoms at any point of the hydrocarbon ring, and wherein two hydrogen atoms are removed from any two carbon atoms of the ring.

As used herein, the term “arylene” refers to an aromatic ring system wherein two hydrogen atoms were removed thus having two open valencies for bonding. For example, a phenylene or a phenylene ring system fused to one or more aromatic rings to form, for example, derivatives of anthracene, phenanthrene, or napthalene ring systems.

The term “heteroarylene” refers to an aromatic ring system wherein at least one of the carbon atoms of the aromatic ring system is replaced by a heteroatom (N, O, P, S) and wherein two hydrogen atoms were removed thus having two open valencies for bonding.

The present invention relates to pharmaceutical compositions comprising a compound of the subject invention in admixture with pharmaceutically acceptable auxiliaries, and optionally other therapeutic agents. The auxiliaries must be “acceptable” in the sense of being compatible with the other ingredients of the composition and not deleterious to the recipients thereof.

Pharmaceutical compositions include those suitable for oral, rectal, nasal, topical (including transdermal, buccal and sublingual), vaginal or parenteral (including subcutaneous, intramuscular, intravenous and intradermal) administration or administration via an implant. The compositions may be prepared by any method well known in the art of pharmacy.

Such methods include the step of bringing in association compounds used in the invention or combinations thereof with any auxiliary agent. The auxiliary agent(s), also named accessory ingredient(s), include those conventional in the art, such as carriers, fillers, binders, diluents, disintegrants, lubricants, colorants, flavoring agents, anti-oxidants, and wetting agents.

Pharmaceutical compositions suitable for oral administration may be presented as discrete dosage units such as pills, tablets, dragées or capsules, or as a powder or granules, or as a solution or suspension. The active ingredient may also be presented as a bolus or paste. The compositions can further be processed into a suppository or enema for rectal administration.

The invention further includes a pharmaceutical composition, as hereinbefore described, in combination with packaging material, including instructions for the use of the composition for a use as hereinbefore described.

For parenteral administration, suitable compositions include aqueous and non-aqueous sterile injection. The compositions may be presented in unit-dose or multi-dose containers, for example sealed vials and ampoules, and may be stored in a freeze-dried (lyophilised) condition requiring only the addition of sterile liquid carrier, for example water, prior to use. For transdermal administration, e.g. gels, patches or sprays can be contemplated. Compositions or formulations suitable for pulmonary administration e.g. by nasal inhalation include fine dusts or mists which may be generated by means of metered dose pressurized aerosols, nebulizers or insufflators.

The exact dose and regimen of administration of the composition will necessarily be dependent upon the therapeutic or nutritional effect to be achieved and may vary with the particular formula, the route of administration, and the age and condition of the individual subject to whom the composition is to be administered.

As used herein, the term “effective amount” means that amount of a drug or pharmaceutical composition that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, slowing the progression of, or a decrease in the rate of advancement of a disease or disorder, condition or symptom. The term also includes within its scope amounts effective to enhance normal physiological function.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 shows the synthetic procedure for the preparation of 1,7-diaminooxy-heptane (VL-849). Hydrogen atoms were not depicted in the molecular representation of the compound for technical reasons, however the compound is fully represented by its molecular name.

FIG. 2 shows the synthetic procedure for the preparation of 1,9-diaminooxy-nonane (VL-851). Hydrogen atoms were not depicted in the molecular representation of the compound for technical reasons, however the compound is fully represented by its molecular name.

FIG. 3 shows the synthetic procedure for the preparation of 1,7-diguanidinoheptane (VL-630). Hydrogen atoms were not depicted in the molecular representation of the compound for technical reasons, however the compound is fully represented by its molecular name.

FIG. 4 shows the synthetic procedure for the preparation of 11,8-diguanidinooctane (VL-640). Hydrogen atoms were not depicted in the molecular representation of the compound for technical reasons, however the compound is fully represented by its molecular name.

FIG. 5 shows the synthetic procedure for the preparation of 1,8-dihydrazine-octane (VL-800). Hydrogen atoms were not depicted in the molecular representation of the compound for technical reasons, however the compound is fully represented by its molecular name.

FIGS. 6A-6B. FIG. 6A shows the synthetic procedure for the preparation of 1,8-diaminooxy-octane (VL-850) and FIG. 6B is the synthetic procedure for the preparation of 1,4-phenyl-bis-butylamine (VL-471). Hydrogen atoms were not depicted in the molecular representation of the compound for technical reasons, however the compound is fully represented by its molecular name.

FIG. 7 shows that 1,8-Diaminooctane of the invention, significantly increases the resistance to paraquat toxicity in a dose-dependent manner. Worms were incubated with increasing concentrations of 1,8-Diaminooctane (0.0625, 0.25, 1, and 4 mM) from the L1 larval stage to the young-adult stage. The control plates contained the same amount of distilled water as the experimental plates. Young adult worms were put in M9 buffer containing 200 mM paraquat (PQ) or in M9 buffer as a control. The survival of the worms was measured after 3, 6, 9, and 24 h incubation at 21° C. Statistical analysis on PQ survival curves were performed using the log-rank (Mantel-Cox) test with Prism 7 software. The significant scores are shown on the right side of each graph and indicate significance for comparisons with control worms that were exposed to PQ without a 1,8-Diaminooctane treatment. The plots represent the average of six independent experiments. The minimum number of worms examined for each experimental condition is 164.

FIGS. 8A-8F. (FIG. 8A) is a schematic illustration of the Mito-Rosella sensor function. The Mito-Rosella sensor contains an N-terminal mitochondrial targeting signal, a pH-stable red fluorescent protein and a C-terminal pH-sensitive green fluorescent protein. In non-acidic environment the green to red fluorescent ratio is 0.6-0.8. However, in the autolysosome, where the pH is low, the green fluorescent is quenched and the green to red ratio is decreased to 0.2-0.5. By measuring the mitochondria red and green fluorescence, it was possible to monitor mitophagy in live worms. (FIG. 8B) shows the in vivo imaging of mitophagy in C. elegans muscles. A bar graph presenting the results of mitophagy imaging experiments. L4 larval worms were treated with either 8 mM paraquat or 1 mM 1,8-Diaminooctane for 16 h, at 21° C. Control worms were treatment with similar volume of distilled water and incubated under similar conditions. Asterisks indicate significance for comparisons with control worms. Data represent the average of at least three independent experiments in which at least 30 worms were imaged for each condition. Error bars represent SEM. One-way ANOVA and Dunnett's posttest. **p<0.01. (FIGS. 8C-8F) Representative images of worms treated with 1,8-Diaminooctane in different light projections: (FIG. 8C)—Bright field, (FIG. 8D)—DsRed, (FIG. 8E)—pHluorion, (FIG. 8F)—merge. Scale bar: 50 m.

FIGS. 9A-9D show the protective activity of 1,8-Diaminooctane is mitophagy-dependent. (FIG. 9A) is a schematic illustration of the mitophagy pathway in C. elegans. Survival curves presenting the effect of dct-1 and pink-1 deletion mutations (FIG. 9B, FIG. 9C) or RNAi knockdowns (FIG. 9D) on worms' viability in 200 mM PQ at indicated time points. Statistical analysis on PQ survival curves were performed using the log-rank (Mantel-Cox) test with Prism 7 software. The plots represent the average of six independent experiments. The minimum number of worms examined for each experimental condition is 133.

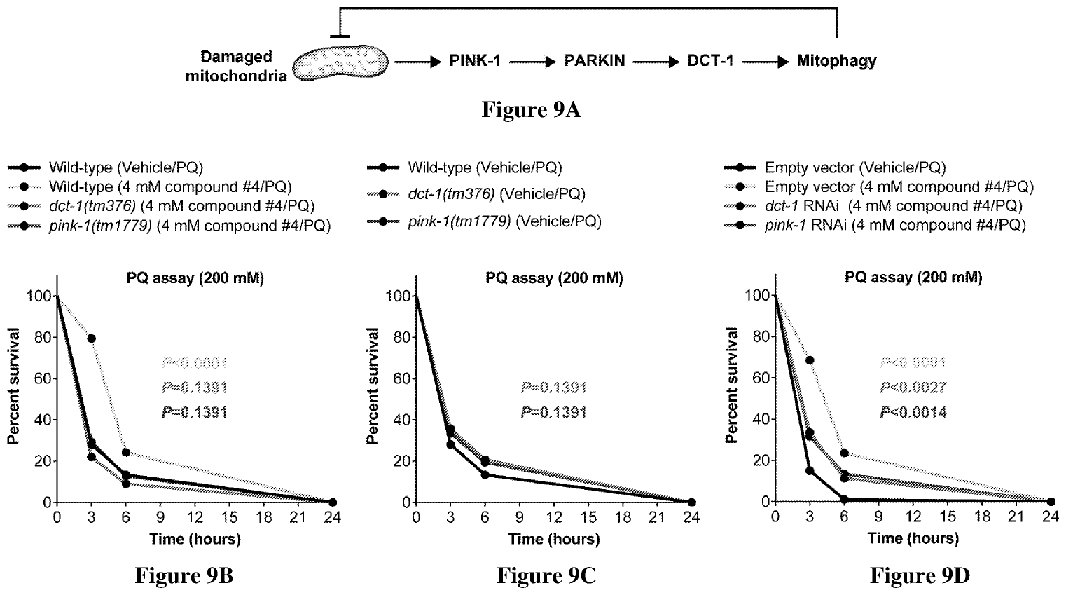

FIG. 10. 1,8-Diaminooctane lengthen the median lifespan of worms, significantly. Survival curves comparing the lifespans of worm growing on plates containing 0.25 mM and 4 mM 1,8-Diaminooctane (or vehicle as a control). The curves represent data from at least three independent experiments in which the lifespans of at least 145 worms were measured (for each condition). The lifespan medians and p values are indicated in the parentheses near the treatment legend. All lifespan assays were conducted at 21° C. and started with synchronized L1 larvae. Statistical analysis on lifespan survival curves was performed using the log-rank (Mantel-Cox).

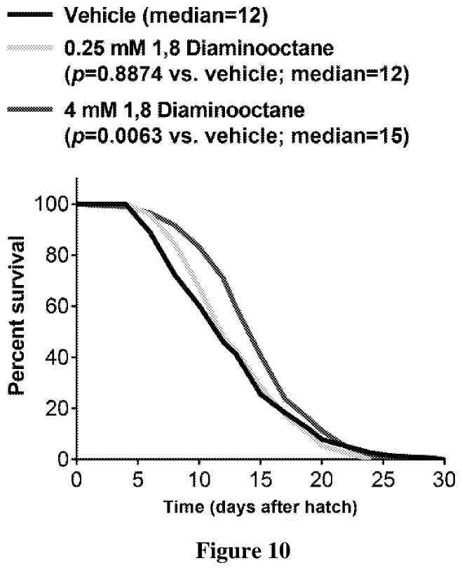

FIG. 11 shows 1,8-Diaminooctane significantly improves locomotion activity in aged worms. While locomotion speed of aging worms (day 11) of the control group dropped by more than 50%, compared to that of young worms (day 3), there is no change in the speed of 1,8-diaminooctane treated worms, with age. Bar graphs presenting the speed of 3 and 11 days post L1 wild-type worms that were treated with 4 mM 1,8-Diaminooctane or with vehicle as a control. The measurements of speed were performed in the absence of food (n=6 assays performed over at least 3 days). Asterisks indicate significance for comparisons with control worms (day 11). **p<0.01, two-way ANOVA with Bonferroni post-test. NS, Not significant. Error bars indicate SEM.

FIGS. 12A-12C show long chain diamine compounds of the invention and their significant protective potency against PQ toxicity. (FIGS. 12A-12C) Survival curves presenting the viability of worms in 200 mM PQ at indicated time points. Statistical analysis on PQ survival curves were performed using the log-rank (Mantel-Cox) test with Prism 7 software. The plots represent the average of six independent experiments. The minimum number of worms examined for each experimental condition is 123. The p values are indicated in the parentheses near the treatment legend.

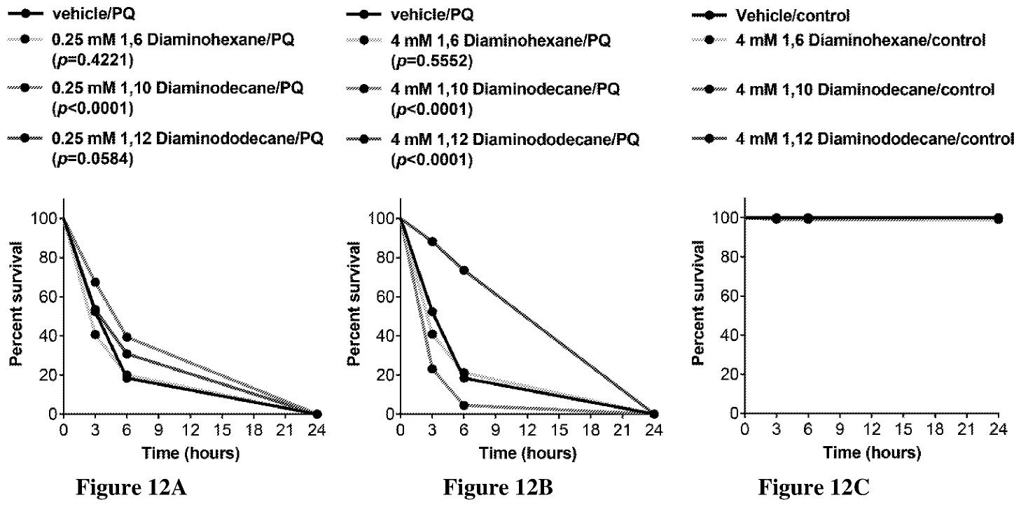

FIG. 13 shows 1,6-Diguanidinohexane (DGH) significant protective effect on worm's survival in PQ. Survival curves presenting the viability of worms in 200 mM PQ at indicated time points. Statistical analysis on PQ survival curves were performed using the log-rank (Mantel-Cox) test with Prism 7 software. The plots represent the average of six independent experiments. The minimum number of worms examined for each experimental condition is 154.

FIG. 14 shows the significant protective effect of 1,9-diaminooxy-nonane on worms' survival in PQ (200 mM) at indicated time points. Statistical analysis on PQ survival curves were performed using the log-rank (Mantel-Cox) test with Prism 7 software. The plots represent the average of six independent experiments. The minimum number of worms examined for each experimental condition is 117.

FIG. 15 shows the significant protective effect of 1,8-diaminooxy-octane on worms' survival in PQ (200 mM) at indicated time points. Statistical analysis on PQ survival curves were performed using the log-rank (Mantel-Cox) test with Prism 7 software. The plots represent the average of six independent experiments. The minimum number of worms examined for each experimental condition is 168.

FIG. 16 shows the significant protective effect of low dose 1,8-diguanidinooctane, compared with low dose of 1,8-diaminooctane, on worms' survival in PQ (200 mM) at indicated time points. Statistical analysis on PQ survival curves were performed using the log-rank (Mantel-Cox) test with Prism 7 software. The plots represent the average of six independent experiments. The minimum number of worms examined for each experimental condition is 119.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

The inventors of the present application have found that a reliable model for testing the activity of the compounds of the present invention are C. elegans worms and their action against paraquat-induced oxidative-injury. The C. elegans model is one of the best-characterized organisms, having outstanding similarity to the human genome (e.g. see Aboobaker A A and Blaxter M L, Medical Significance of C. elegans, Ann. Med. 32: 23-30 (2000)). Many key molecular and physiological processes, that underlie autophagy, mitophagy, wellness and lifespan, are shared by humans and C. elegans and it has been proven to be an excellent model organism for exploring the molecular mechanisms underlying neurodegenerative diseases development such as Parkinson's and Alzheimer's and including a variety of age-related diseases such as congestive heart failure and sarcopenia.

Moreover, recent development in imaging platforms and data analysis software have paved the way for high-throughput drug discovery in C. elegans as preferred organism in drug discovery for aging associated neurodegenerative diseases (Chen X. et al. Chem Sent J (2015) 9:65).

Chemical Synthesis of Compounds of the Invention

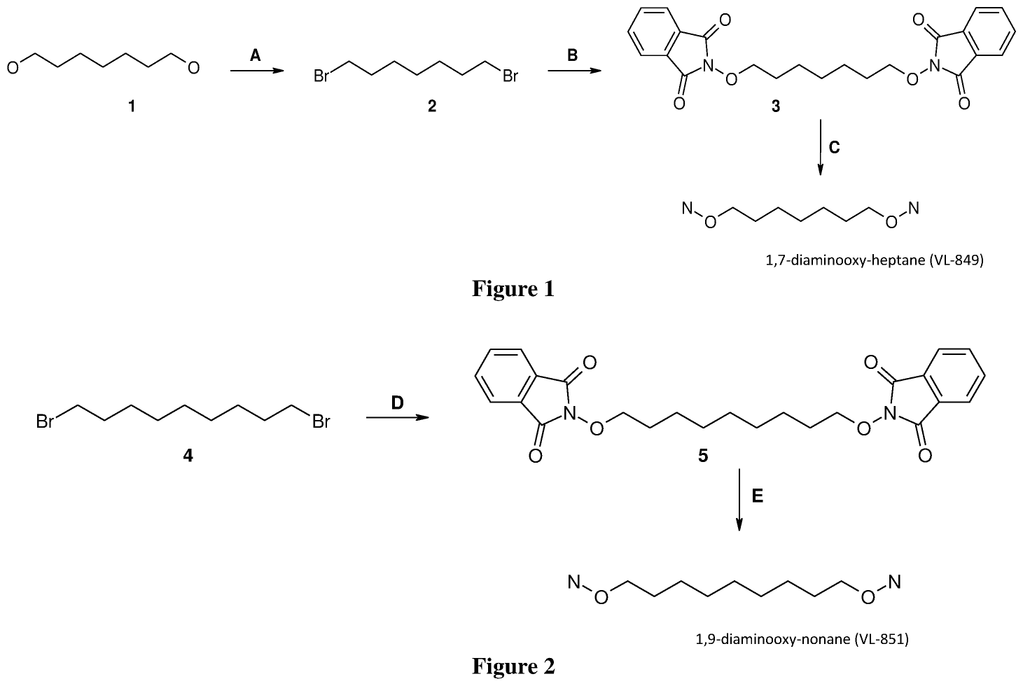

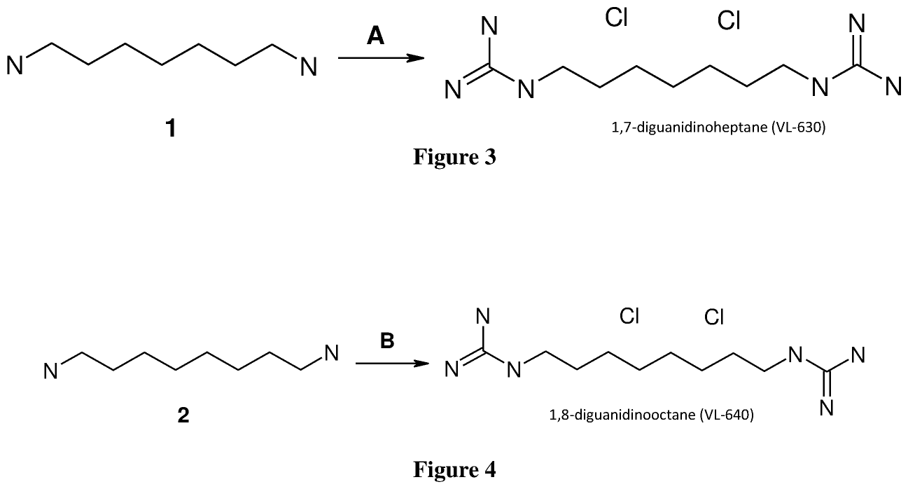

### Example 1: Synthetic Procedure for the Preparation of 1,7-diaminooxy-heptane (VL-849) and 1,9-diaminooxy-nonane (VL-851) (Synthetic Schemes in FIGS. 1 and 2)

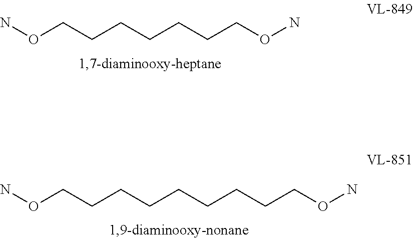 [SMILES: NOCCCCCCCON ; NOCCCCCCCCCON]

Hydrogen atoms were not depicted in the molecular representation of the compound for technical reasons, however the compound is fully represented by its molecular name.

Step A: To a solution of compound 1 (80.0 g, 605.0 mmol) in diethyl ether (1000 mL) two drops of triethylamine were added with stirring at 0° C. PBr₃ (75.0 g, 277.1 mmol) was added over 1.5 hours and the reaction mixture was left overnight with stirring. Then the mass was poured onto ice (200.0 g), the ether layer was evaporated. The residue was distilled in an oil pump vacuum to give 75.0 g (291.0 mmol, 48%) of compound 2.

Step B: To a mixture of 2-hydroxyphthalimide (95.0 g, 582.4 mmol) and triethylamine (73.5 g, 726.4 mmol) in DMF (800 mL) the solution of compound 2 (75.0 g, 291.0 mmol) in DMF (200 mL) was added. The reaction mixture was left with stirring at room temperature for 72 hours. After a mass was poured into water (3000 mL). The precipitate was filtered, washed with water and methyl tert-butyl ether, dried in the air to give 100.0 g (236.7 mmol, 81%) of compound 3.

Step C: To a solution of compound 3 (100.0 g, 236.7 mmol) in THF (1200 mL) at −10-15° C. 40% aqueous methylhydrazine (70 mL) was added during 10 min. The reaction mixture was left with stirring at room temperature for 2 hours. The precipitate was filtered, washed with THF. The mother liquor was evaporated, hexane (300 mL) was added to the residue and filtered. The hexane was evaporated, the residue was distilled in an oil pump vacuum at 85-90° C. to give 28.0 g (172.6 mmol, 73%) of compound VL-849.

Step D: To a mixture of 2-hydroxyphthalimide (80.0 g, 490.4 mmol) and triethylamine (62.0 g, 612.7 mmol) in DMF (700 mL) the solution of compound 4 (80.0 g, 279.6 mmol) in DMF (200 mL) was added. The reaction mixture was left with stirring at room temperature for 72 hours. After a mass was poured into water (3000 mL). The precipitate was filtered, washed with water and methyl tert-butyl ether, dried in the air to give 75.0 g (166.5 mmol, 60%) of compound 5.

Step E: To a solution of compound 5 (75.0 g, 166.5 mmol) in THF (1200 mL) at −10-15° C. 40% aqueous methylhydrazine (57.5 mL) was added during 10 min. The reaction mixture was left with stirring at room temperature for 2 hours. The precipitate was filtered, washed with THF. The mother liquor was evaporated, hexane (300 mL) and methyl tert-butyl ether (100 mL) were added to the residue and filtered. The hexane and the methyl tert-butyl ether were evaporated, the residue was distilled in an oil pump vacuum at 105-110° C. (at 115° C. decomposed) to give 16.0 g (84.1 mmol, 50%) of compound VL-851.

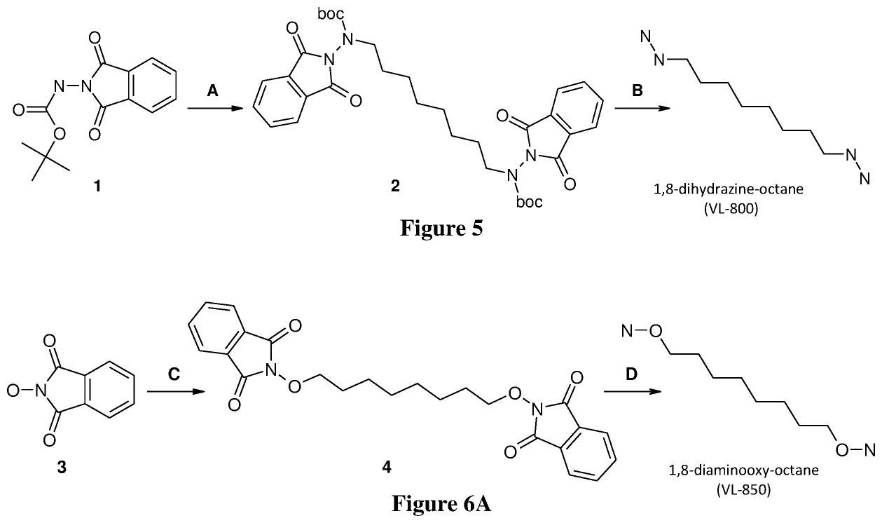

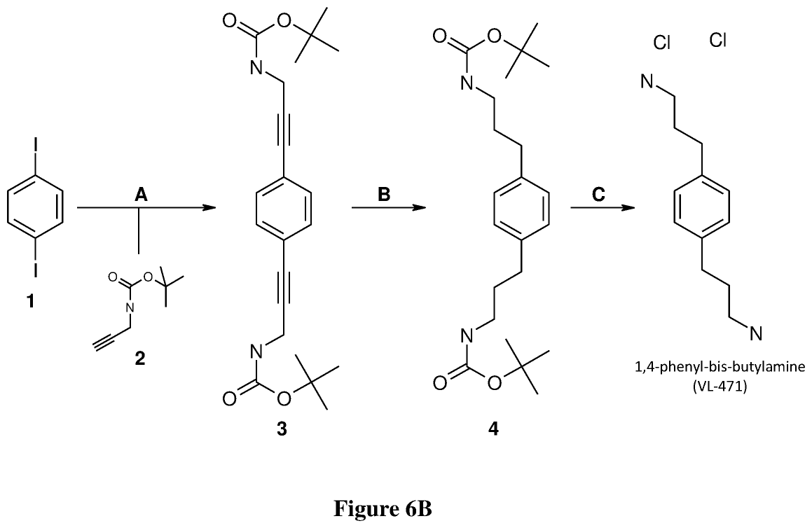

### Example 2: Synthetic Procedure for the Preparation of 1,7-diguanidinoheptane (VL-630) and 1,8-diguanidinooctane (VL-640) (Synthetic Schemes in FIG. 3 and FIG. 4)

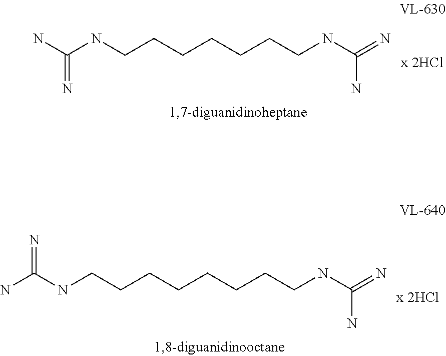 [SMILES: N=C(N)NCCCCCCCNC(=N)N ; N=C(N)NCCCCCCCCNC(=N)N]

Hydrogen atoms were not depicted in the molecular representation of the compound for technical reasons, however the compound is fully represented by its molecular name.

Step A: To a mixture of compound 1 (4.53 g, 34.8 mmol), acetonitrile (200 mL), and DMF (50 mL) DIPEA (9.00 g, 69.6 mmol) and pyrazole-1-carboxamidine hydrochloride (10.5 g, 71.6 mmol) were added and the reaction mass was left to stir at r.t. for 5 days. Then, the precipitated solid was collected by filtration, washed with acetonitrile, and recrystallized from ethanol to give 4.70 g (16.4 mmol, 47%) of target compound VL-630.

Step B: To a mixture of compound 2 (4.79 g, 33.2 mmol), acetonitrile (200 mL), and DMF (50 mL) DIPEA (8.58 g, 66.4 mmol) and pyrazole-1-carboxamidine hydrochloride (9.97 g, 68.0 mmol) were added and the reaction mass was left to stir at r.t. for 5 days. Then, the precipitated solid was collected by filtration, washed with acetonitrile, and recrystallized from ethanol to give 5.30 g (17.6 mmol, 53%) of target compound VL-640.

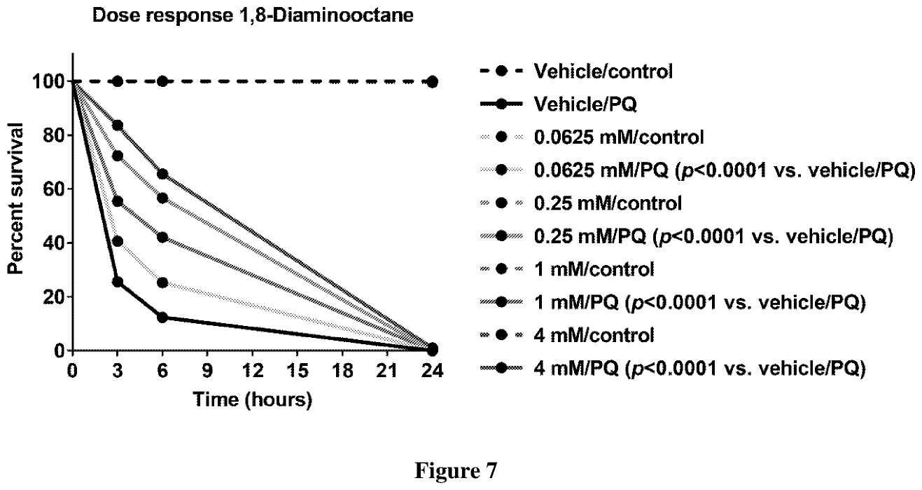

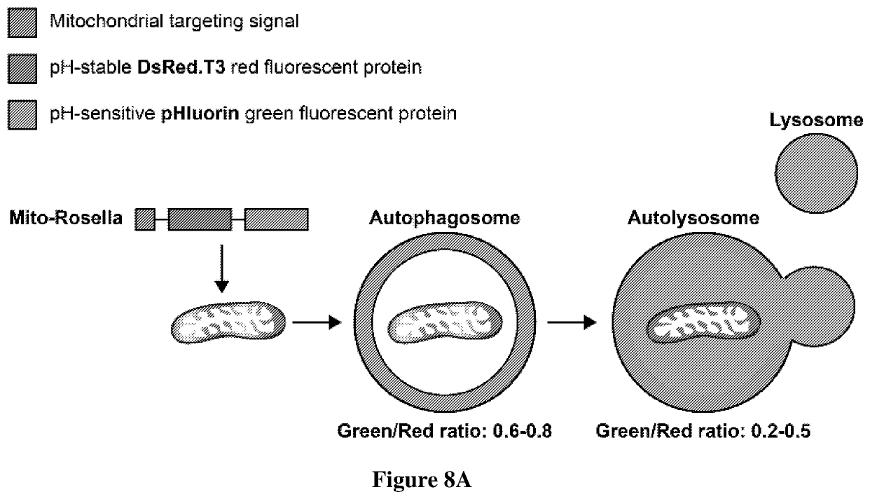

### Example 3: Synthetic Procedure for the Preparation of 1,8-dihydrazine-octane (VL-800) and 1,8-diaminooxy-octane (VL-850) (Synthetic Schemes in FIG. 5 and FIG. 6A)

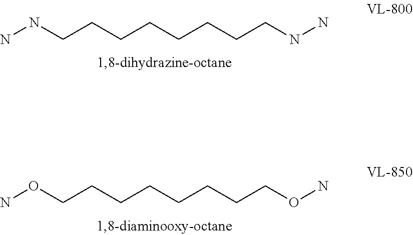 [SMILES: NNCCCCCCCCNN ; NOCCCCCCCCON]

Hydrogen atoms were not depicted in the molecular representation of the compound for technical reasons, however the compound is fully represented by its molecular name.

Step A: To a suspension of NaH (10.0 g, 250 mmol) in DMF (100 ML) a solution of compound 1 (61.0 g, 233 mmol) in DMF (200 mL) was added dropwise and the reaction mass was stirred of 0.5 h at r.t. Then, a solution of 1,8-dibromooctane (31.3 g, 115 mmol) in DMF (50 mL) was added and the resulting mixture was stirred for 16 h at r.t. The obtained solution was poured into water (1000 mL). The precipitated solid was collected by filtration, washed with water (3×300 mL) and hexane (2×100 mL), and dried to obtain 62.0 g (89% yield) of compound 2.

Step B: To a cooled to 10° C. stirring solution of compound 2 (62.0 g, 97.7 mmol) in THF (600 mL) 40% aqueous methylhydrazine (34 mL) was added dropwise over 10 min and the reaction mass was stirred for 12 h at r.t. The precipitated solid was filtered off and rinsed with THF (2×100 mL). The filtrate and rinses were evaporated under reduced pressure and the residue was mixed with hexane. The insoluble solid was filtered off and the filtrate was evaporated under reduced pressure. The obtained material was dissolved in methanol (100 mL) and added dropwise to a heated to 45° C. mixture of methanol (100 mL) and concentrated hydrochloric acid (50 mL). The resulting mixture was stirred at 45° C. for 2 h and then evaporated to dryness under reduced pressure. The residue was recrystallized from ethanol to obtain 17.5 g (73% yield) of target compound VL-800 as dihydrochloride salt.

Step C: To a mixture of compound 3 (18.0 g, 110 mmol) and triethylamine (14.0 g, 138 mmol) in DMF (150 mL) a solution of 1,8-dibromooctane (15.0 g, 55.0 mmol) in DMF (20 mL) was added and the reaction mass was stirred for 72 h at r.t. The obtained solution was poured into water (500 mL). The precipitated solid was collected by filtration, washed with water (3×100 mL) and MTBE (2×50 mL) and air-dried to obtain 14.0 g (58% yield) of compound 4.

Step D: To a cooled to 10° C. stirring solution of compound 3 (14.0 g, 85.8 mmol) in THF (200 mL) 40% aqueous methylhydrazine (11 mL) was added and the reaction mass was stirred for 12 h at r.t. The precipitated solid was collected by filtration and rinsed with THF (2×50 mL). The filtrate and rinses were evaporated under reduced pressure and the residue was mixed with hexane (100 mL). The insoluble solid was filtered off and the filtrate was evaporated in vacuo. The residue was purified by vacuum distillation to obtain 3.80 g (67% yield) of target compound VL-850.

### Example 4: Synthetic Procedure for the Preparation of 1,4-phenyl-bis-butylamine (VL-471) (Synthetic Schemes in FIG. 6B)

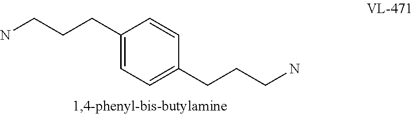 [SMILES: NCCCc1ccc(CCCN)cc1]

Hydrogen atoms were not depicted in the molecular representation of the compound for technical reasons, however the compound is fully represented by its molecular name.

Step A: Under argon atmosphere, to a solution of compound 1 (18.36 g, 55.7 mmol), CuI (1 g, 5.25 mmol), and Pd(PPh₃)₄ (3.13 g, 2.71 mmol) in dry CH₃CN (100 mL) was added a solution of compound 2 (25.7 g, 166 mmol) in dry NEt₃ (14 mL). The reaction mixture was stirred under argon atmosphere for 2 days. Then diluted with CH₂Cl₂, washed with water, and concentrated under reduced pressure. Reduced-pressure column chromatography (hexanes:EtOAc 15:1) of the residue gave 3 g of compound 3 (7.80 mmol, 14% yield) as a white solid.

Step B: Compound 3 (2.4 g, 6.24 mmol) was dissolved in methanol (60 mL) and treated with 10% Pd(OH)₂(C) (0.24 g). The resulting mixture was hydrogenated at 20 bar and room temperature until the reaction was complete (TLC control). The catalyst was filtered off and the filtrate was evaporated to afford 2.3 g of compound 4 (5.86 mmol, 94% yield).

Step C: Compound 4 (2.3 g, 5.86 mmol) was dissolved in methanol (50 mL) and 4M HCl:dioxane (10 mL) at r.t. The resulting mixture was stirred overnight. Upon completion of the reaction (monitored by HNMR), the resulting mixture was evaporated to dryness to obtain 1.5 g of target compound VL-471 (5.65 mmol, 96% yield) as solid residue.

Biological Experimental Procedures

C. elegans Strains and NGM-Plate Preparation:

C. elegans strains were grown at 21° C. on nematode growth media (NGM) agar plates containing diamines or vehicle as a control. The NGM agar plates were prepared as described in L. Livshits, E. Gross, A method for measuring sulfide toxicity in the nematode Caenorhabditis elegans, MethodsX 4 (2017) 250-255. A suspension of 3 g sodium chloride (NaCl), 20 g Bacto agar, and 2.5 g of Bacto peptone in 1 L of double distilled water (DDW) was mixed and autoclaved. Then, the medium was cooled to 55° C. and supplemented with 1 ml of 100 mM CaCl₂), 1 ml of 100 mM MgSO4, 25 ml of 1 M potassium phosphate buffer pH 6, and 1 ml of 5 mg/ml cholesterol (the cholesterol was dissolved in ethanol). Diamines or DDW (vehicle) were added to the NGM-agar solution. For RNAi NGM-agar plates, Isopropyl β-D-1-thiogalactopyranoside (IPTG) and ampicillin were added to a final concentration of 1 mM and 100 μg/ml, respectively. The NGM-agar solution was thoroughly vortex mixed and immediately poured into 35 mm petri dish (4 ml per plate). NGM-plates were covered with aluminum foil and dried for 24 h at room temperature (RT). 24 h before the experiment, plates were seeded with 100 μl bacteria (OD₆₀₀=0.6, OP50 or HT115(DE3)). The seeded plates were covered with aluminum foil and dried for 24 h at RT.

Bacteria Preparation:

To Make Luria-Bertani (LB) agar plates, 10 g NaCl, 10 g Bacto tryptone, 5 g Bacto yeast extract, and 15 g Bacto agar were dissolved in 1 litter of DDW, autoclave, cool to 55° C., and pour 25 ml per 9 cm petri dish. LB-agar plates with ampicillin were made by adding 1 ml of 100 mg/ml ampicillin (final concentration of 100 μg/ml). LB plates were dried at RT for two days prior to use. To Make 2× yeast tryptone (YT) medium, 5 g NaCl, 16 g bacto tryptone, and 10 g bacto yeast extract were dissolved in 1 litter of DDW and adjust the pH to 7. Autoclave and let cool to RT. For RNAi bacteria (HT115(DE3)), ampicillin was added to a final concentration of 100 μg/ml. To grow bacteria, the LB plates were streaked with bacteria (OP50 or HT115(DE3)) and incubated at 37° C. overnight (O/N). A single colony was grown in 3 ml of 2XYT in a 15 ml conical tube and shaked O/N at 37° C., 220 rpm. HT115(DE3) bacteria were grown with 100 μg/ml ampicillin. The O/N starter was diluted 200 fold in 2XYT (in a 500 ml Erlenmeyer flask) and Shaked at 37° C., 200 rpm, until an OD₆₀₀ of ˜0.6.

Isolation of Specific Larval Stage:

To get synchronized C. elegans larvae, the method described in W. B. Wood, The Nematode Caenorhabditis Elegans, Cold Spring Harbor Laboratory 1988, was performed. In brief, hypochlorite/NaOH solution was made by mixing 1 ml of 5% solution of sodium hypochlorite, 800 μl of 2.5 N NaOH and 2.2 ml DDW to final concentrations of 0.5 N NaOH and 1.25% sodium hypochlorite. M9 buffer was made by dissolving 3 g KH₂PO₄, 6 g Na₂HPO₄, 5 g NaCl and 1 ml of MgSO₄ from 1M stock in 1 litter of DDW. The solution pH was adjusted to 7 and sterilizes by filtration (with a 0.2 m filter bottle). Gravid hermaphrodites was washed from the NGM plates into a 15 ml tube with M9 buffer and centrifuged for 2 min at 900×g, RT. Liquid was removed until 2 ml worms' suspension was left and added 2 ml of hypochlorite/NaOH solution. A 5 ml syringe with a 21-gauge needle was used to aspirate the worm suspension back and forth several times. After 3 min it was observed the state of the worms using a dissecting stereoscope. At this stage, approximately 50% of worms should appear broken and many of the embryos should float in the solution. The embryos were immediately sedimented using centrifugation (1690×g for 2 min) and carefully removed the supernatant and add 10 ml of M9 buffer. This washing step was repeated three additional times. The supernatant was removed until 2 ml remains and rotated the tube for 16 h at RT. The hatched L larvae were collected by centrifugation (1690×g for 3 min) and put ˜80 L in each seeded NGM assay plate. Grow the L1 (Day 1) for 3 days until they become young adults (Day 3).

Paraquat Survival Assay:

Paraquat (PQ) survival assays were performed as described in L. Livshits, A. K. Chatterjee, N. Karbian, R. Abergel, Z. Abergel, E. Gross, Mechanisms of defense against products of cysteine catabolism in the nematode Caenorhabditis elegans, Free radical biology & medicine 104 (2017) 346-359. In brief, the worms (day 3) were collected from the NGM-agar plate by washing them with M9 buffer. Two additional washes were performed in order to remove bacteria. To assay PQ toxicity, ˜12 worms were put in a well (in 96-well plate) containing 100 μl of 200 mM Paraquat (in M9 buffer) or to M9 buffer as a control. The plate was shaken at 350 rpm on an orbital shaker at RT. Worm's survival was measured after 3, 6, and 24 h by touching them with an eyelash. In general, six independent assays for each strain/RNAi/treatment were performed. The total number of worms for each experiment was at least 120.

Off-Food Speed Measurements:

For speed-imaging experiments, low-peptone NGM agar plates were used. These plates were prepared as described above apart from the of Bacto peptone concentration that was decreased to 0.13 g/L and the use of 60 mm petri dished (instead of 35 mm dishes). Worms speed was measured as described in L. Livshits, A. K. Chatterjee, N. Karbian, R. Abergel, Z. Abergel, E. Gross, Mechanisms of defense against products of cysteine catabolism in the nematode Caenorhabditis elegans, Free radical biology & medicine 104 (2017) 346-359. In brief, eight synchronized worms (age 3 or 11 post-L1) were put (for 30 min) in a drop of M9 buffer to clean them from external and internal bacteria. During the 30 min the M9 buffer was replaced twice. After 30 min, the worms were placed at the center of a 17 mm diameter copper ring on an unseeded low-peptone plate. Recording was started after 5 min acclimation time. The worms were recorded for 10 min at 0.5 frames/s, using a Q-Imaging MicroPublisher 5.0 RTV Microscope Camera mounted onto an Olympus SZ61 stereo microscope. A custom-written MATLAB software was used to analyze the speed. For each treatment, at least 48 animals in 6 independent assays were measured.

Lifespan Assay:

Lifespan assays were performed as described in R. Abergel, L. Livshits, M. Shaked, A. K. Chatterjee, E. Gross, Synergism between soluble guanylate cyclase signaling and neuropeptides extends lifespan in the nematode Caenorhabditis elegans, Aging cell 16 (2) (2017) 401-413. In brief, 12 worms per plate were used. Notably, these worms were exposed to diamine or vehicle from the L1 stage (day 0). Worm's survival was scored every two days for live, dead (when it no longer responded to touch), and missing worms. The worms were transferred into fresh plates every 2 days to avoid progeny contamination and every 4 days when worms are in post-fertile stage, until the end of the experiment. Worms were scored as censored when they display internal progeny hatching (worm bagging), rupture, burrow in the agar or crawl off the plates, however, include them in the lifespan data analysis as censored. Every biological set included ˜60 worms and at least total number of 120 animals for each treatment/condition. All life span studies were performed at 21° C.

Mitophagy Imaging Experiments:

One day before the imaging experiment, ˜20 L4 of Rosella worms (wild-type N2; Ex003[Pmyo-3TOMM-20::Rosella]) were placed on NGM-agar plates containing diamines or vehicle as a control for 24 hours at 21° C. For positive control PQ was used as described in K. Palikaras, E. Lionaki, N. Tavernarakis, Coordination of mitophagy and mitochondrial biogenesis during ageing in C. elegans, Nature 521 (7553) (2015) 525-8. In brief, regular NGM plates were seeded with 100 μl OP50. In the next day, the plates were UV irradiated for 15 min (0.5 J) using a UV crosslinker. To these plates, 64 μl of PQ (500 mM stock solution in DDW) were added to a final concentration of 8 mM and let it diffuse for 4 h in RT. Then, ˜20 L4 Rosella worms were transferred to these plates and kept them at 21° C. for 24 hours. To make agarose pads, 2% agarose M9 buffer solution was made with Ethyl 3-aminobenzoate methanesulfonate (Tricaine) and Tetramisole hydrochloride (final concentrations of 0.05% w/v and 15 mM, respectively). Once solidified, 2 μL of the same solution (without the agarose) was placed on the pad, transferred the worms to it, and cover with an 18×18 cover glass. The worms were imaged using an Olympus IX71S1F-3-5 inverted microscope equipped with UPlanFLN10× and Q-Imaging Rolera EM-C2™ camera. Measure GFP and mCharry wavelength in exposure of 150 ms and EM Gain of 3850 using MetaMorph Microscopy Automation and Image Analysis Software. Measure DIC in exposure of 8 ms and EM Gain of 0. Perform image analysis using ImageJ.

Strains:

N2 (wild-type), dct-1 (tm376), pink-1 (tm1779), N2; Ex003[Pmyo-3TOMM-20:Rosella]

Materials

Ampicillin sodium salt (Sigma, Cat. No. A9518)
Bacto agar (BD-Difco, Cat. No. 214010)
Bacto peptone (BD-Difco, Cat. No. 211677)
Bacto tryptone (BD-Difco, Cat. No. 211705)
Bacto yeast extract (BD-Difco, Cat. No. 212750)
Calcium chloride (Sigma, Cat. No. C1016)
Cholesterol (Sigma, Cat. No. C8667)
Dibasic potassium phosphate (Sigma, Cat. No. P3786)
Double distilled water (DDW)
Ethyl 3-aminobenzoate methanesulfonate (Sigma, Cat. No. E10521)
Magnesium sulfate (Sigma, Cat. No. M2670)
Methyl Viologen hydrate (Sigma, Cat. No. 856177)
Olympus IX71S1F-3-5 inverted microscope (Olympus)
Olympus SZ61 stereo microscope (Olympus)
Potassium dihydrogen phosphate (Merck, Cat. No. 1.04873.1000)

### Q-Imaging MicroPublisher 5.0 RTV Microscope Camera (QImaging, RHos)

Q-Imaging Rolera EM-C2™ camera (QImaging, RHos)
SeaKem® LE agarose (Lonsa, Cat. No. 50004)
Sodium chloride (Bio-Lab Cat. No. 0011903059100)
Sodium hydroxide (Gadot, Cat. No. 830224310)
Ultrospec 10 Cell density meter (biochrom)

### UPlanFLN10× (Olympus)

Tetramisole hydrochloride (Sigma, Cat. No. L9756)

### Example 4: 1,8-Diaminooctane Protects Against Oxidative Injury Caused by Paraquat

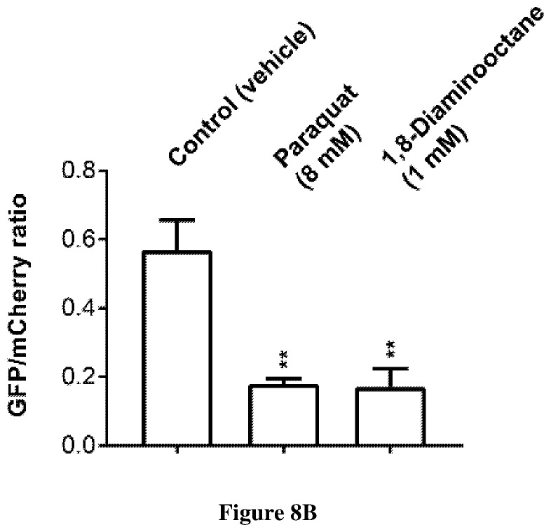

Mitophagy protects against oxidative stress (D. Dutta, J. Xu, J. S. Kim, W. A. Dunn, Jr., C. Leeuwenburgh, Upregulated autophagy protects cardiomyocytes from oxidative stress-induced toxicity, Autophagy 9 (3) (2013) 328-44). Therefore, if 1,8-Diaminooctane induces mitophagy, it should protect against paraquat toxicity. To test this, the worms were treated with increasing concentrations of 1,8-Diaminooctane (or with distilled water as a control) for ˜48 h and monitored their survival in 200 mM paraquat (PQ). All 1,8-Diaminooctane treatments increase the survival of worms significantly (FIG. 7, p<0.0001). However, the highest 1,8-Diaminooctane concentration (4 mM) provided the best protection.

### Example 5: 1,8-Diaminooctane Induces Mitophagy in the Nematode C. elegans

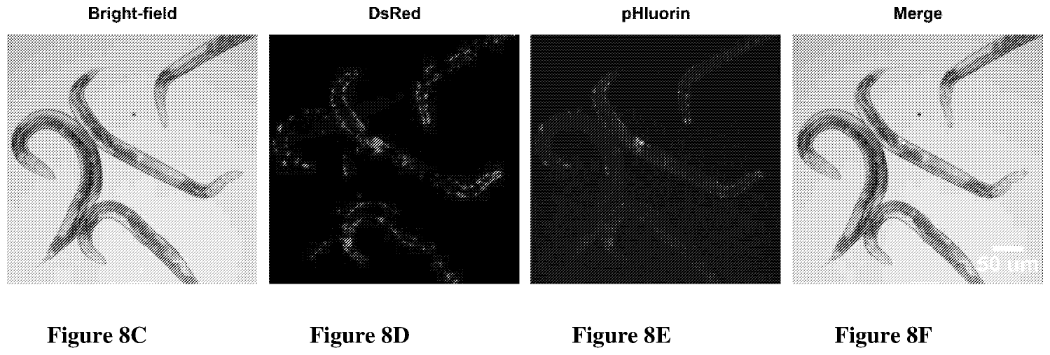

To demonstrate that 1,8-Diaminooctane induces mitophagy in C. elegans the Mito-Rosella genetically encoded sensor was used (see K. Palikaras, E. Lionaki, N. Tavernarakis, Coordination of mitophagy and mitochondrial biogenesis during ageing in C. elegans, Nature 521 (7553) (2015) 525-8). The Mito-Rosella sensor is composed of an N-terminal mitochondrial targeting sequence followed by a pH-insensitive red fluorescent protein (DsRed.T3) and a pH-sensitive green fluorescent protein (pHluorin) (FIG. 8A). In intact mitochondria the green to red fluorescence ratio is high (between 0.6-0.8). However, upon acidification in autolysosome (where mitochondria are degraded) the green fluorescence is quenched and thus the ration is decreased to 0.2-0.5. Exposure of worms to 1 mM 1,8-Diaminooctane generated a strong mitophagy response (FIG. 8B,C). Importantly, the magnitude of the response was similar to the positive paraquat control (FIG. 8B), indicating that 1,8-Diaminooctane is a potent mitophagy inducer.

### Example 6: The Protective Activity of 1,8-Diaminooctane is Mitophagy-Dependent

To prove that the protective activity of 1,8-Diaminooctane is mitophagy-dependent, two experiments were performed. In the first one, the effect of 1,8-Diaminooctane on the survival of dct-1 and pink-1 mutants in PQ was investigated. DCT-1 is an orthologue of the mammalian NIX/BNIP3L and BNIP3 that act as mitophagy receptors, and PINK-1 is a mitochondrial phosphatase and tensin (PTEN)-induced kinase 1 which has a critical function in mitophagy (see K. Palikaras, E. Lionaki, N. Tavernarakis, Coordination of mitophagy and mitochondrial biogenesis during ageing in C. elegans, Nature 521 (7553) (2015) 525-8; and FIG. 3A). It was hypothesized that if mitophagy is essential for the protective effect of 1,8-Diaminooctane, then dct-1 and pink-1 mutants will become sensitive to PQ following treatment. Indeed, worms bearing the dct-1(tm376) and pink-(tm1779) deletion alleles were significantly less resistant to PQ after 1,8-Diaminooctane treatment (compared to treated wild-type worms, FIG. 3B, p<0.0001), suggesting that mitophagy is essential for 1,8-Diaminooctane protective activity. Notably, the survival of control dct-1(tm376) and pink-1(tm1779) mutants in PQ was similar to wild-type worms (FIG. 3C), indicating that the sensitivity of these mutants to PQ (after the 1,8-Diaminooctane treatment) is not due to a general sickness or oversensitivity to oxidative stress. The second experiment that was performed was an RNAi experiment. Here, dct-1 and pink-1 were selectively knocked down using RNAi. The RNAi results recapitulated the one of the mutant experiments (FIG. 3D). The protective effect of 1,8-Diaminooctane was significantly lesser in worms that were treated with RNAi against dct-1 and pink-1 (compared to worms treated with mock RNAi-empty vector). Importantly, the RNAi phenotype was less strong compared to the one observed with the deletion mutants. The sensitivity of the dct-1 and pink-1 mutants to PQ (after the 1,8-Diaminooctane treatment) was similar to the one observed in control wild-type worms (FIG. 3B, p=0.1391 and p=0.9926, respectively), indicating that these genes are absolutely essential for the protective effect. However, although RNAi against dct-1 and pink-1 decrease the effect of 1,8-Diaminooctane, the survival of the treated worms were significantly higher than control worms that did not get the treatment (p<0.0001). The difference between the results could be due to incomplete suppression of dct-1 and pink-1 in the RNAi treated worms. Together, our results verify that 1,8-Diaminooctane protects against oxidative stress by activating mitophagy.

### Example 7: 1,8-Diaminooctane Lengthen the Average Lifespan of Worms

A Previous study showed that mitophagy extends the lifespan of C. elegans (see D. Ryu, L. Mouchiroud, P. A. Andreux, E. Katsyuba, N. Moullan, A. A. Nicolet-Dit-Felix, E. G. Williams, P. Jha, G. Lo Sasso, D. Huzard, P. Aebischer, C. Sandi, C. Rinsch, J. Auwerx, Urolithin A induces mitophagy and prolongs lifespan in C. elegans and increases muscle function in rodents, Nat Med 22 (8) (2016) 879-88). Since 1,8-Diaminooctane induces mitophagy, it was hypothesized that it will also lengthen worms' lifespan. To test it, the lifespan of worms grown on plates containing 0.25 mM and 4 mM 1,8-Diaminooctane (or vehicle as a control) was measured. The 4 mM 1,8-Diaminooctane treatment significantly lengthened worms' median lifespan (FIG. 4, from 12 to 15 days, p=0.0063). This effect was dose-dependent since the lifespan of worms treated with 0.25 mM 1,8-Diaminooctane was similar to control worms (p=0.8874). In conclusion, it was demonstrated that 1,8-Diaminooctane provide protection against acute oxidative stress and extends worms lifespan.

### Example 8: 1,8-Diaminooctane Improves the Locomotory Activity of C. elegans in Old Age

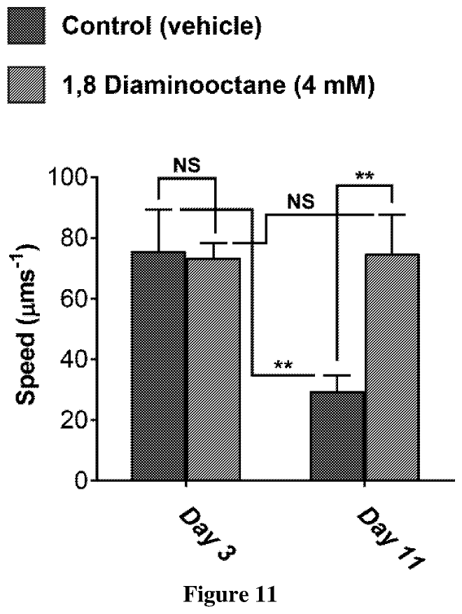

To explore whether 1,8-Diaminooctane improves healthspan, speed measurements in young-adult worms (3 days post L1 stage) and in mid-aged worms (11 days post L stage) were performed. Previous studies showed that short physical performance assays are correlated with healthspan of worms (and humans). For example, long-lived daf-2 mutants maintain high vigor in old age and high speed on plates without food (see J. H. Hahm, S. Kim, R. DiLoreto, C. Shi, S. J. Lee, C. T. Murphy, H. G. Nam, C. elegans maximum velocity correlates with healthspan and is maintained in worms with an insulin receptor mutation, Nat Commun 6 (2015) 8919). On day 3 (post L1), 1,8-Diaminooctane-treated and non-treated worms had similar speed (FIG. 11), indicating that 1,8-Diaminooctane cannot further increase worms vigor in young age. By contrast, in day 11 the speed on the untreated worms declined significantly (p=0.0089 compared to days 3), whereas the speed of the treated worms remained high and was similar to day 3. These results demonstrate that not only 1,8-Diaminooctane extends lifespan and protects against oxidative stress, it also improves healthspan in old age, significantly.

### Example 9: Longer, but not Shorter Chain Diamines Protect Against Paraquat Toxicity

A Previous study showed that long chain diamines (NH₂(CH₂)ₓNH₂; x=9,10,12) have antiproliferative properties, whereas short diamines (NH₂(CH₂)ₓNH₂; x=2-8) do not (see R. Hochreiter, T. M. Weiger, S. Colombatto, T. Langer, T. J. Thomas, C. Cabella, W. Heidegger, M. A. Grillo, A. Hermann, Long chain diamines inhibit growth of C6 glioma cells according to their hydrophobicity. An in vitro and molecular modeling study, Naunyn Schmiedebergs Arch Pharmacol 361 (3) (2000) 235-46). To test whether long chain diamines are also potent against paraquat toxicity, the worms were exposed to 1,10-Diaminodecane and 1,12-Diaminododecane (0.25 and 4 mM) and measured their survival in 200 mM PQ. In addition, a short diamine (1,6-Diaminohexane) at the same concentrations (FIG. 6A, 6B) was explored. The effect of the long chain diamines was dose dependent. At 0.25 mM 1,10-Diaminodecane significantly improved worms' survival (p<0.0001, FIG. 6A), whereas 1,12-Diaminododecane did not. However, at 4 mM, 1,10-Diaminodecane significantly decreased worms' survival (p<0.0001, FIG. 6B), whereas 1,12-Diaminododecane significantly improved their survival (p<0.0001). Notably, these long-chain diamines did not change the viability of worms that are not exposed to PQ (FIG. 6C). On the other hand, 1,6-Diaminohexane did not have any effect on worms viability in PQ (FIGS. 6A, 6B), suggesting that diamines' chain-length plays a crucial role in determining their potency against PQ toxicity.

### Example 10: Compounds of the Invention Protect Against Oxidative Injury Caused by Paraquat

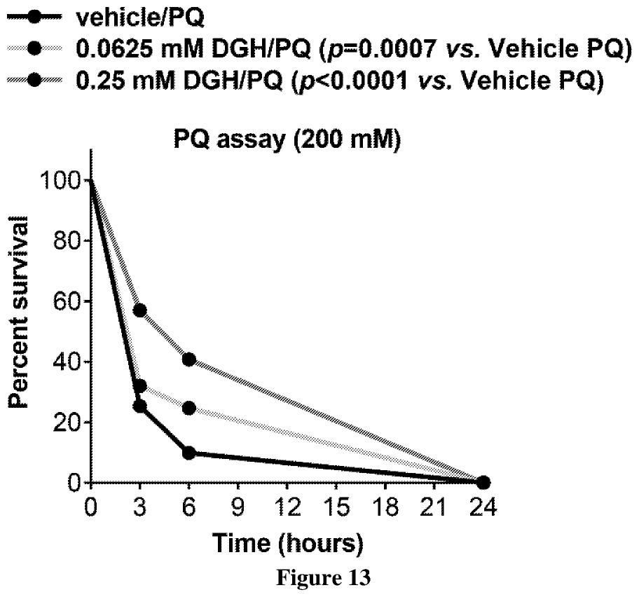

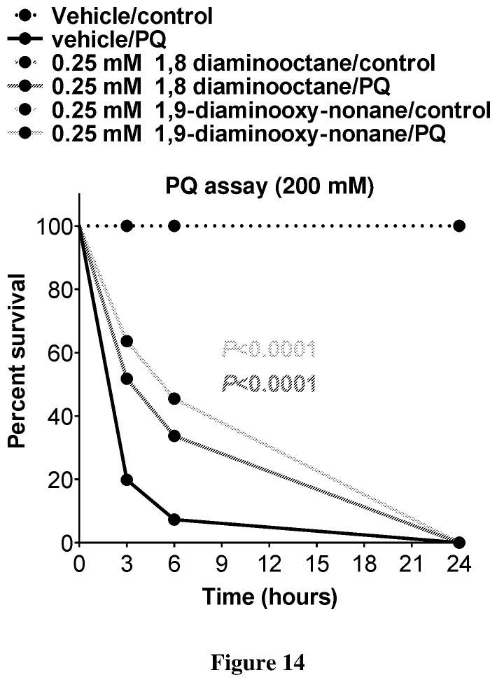

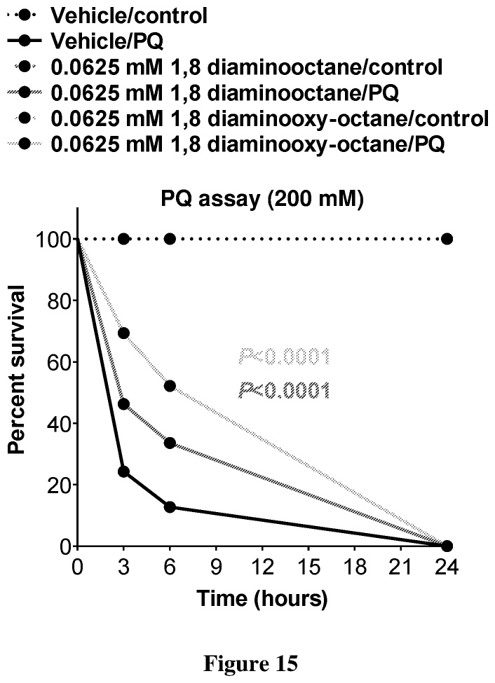

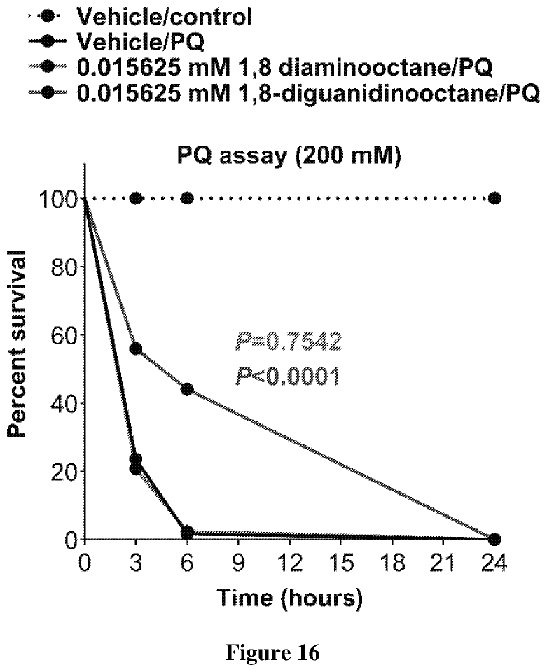

To explore the potency of compounds of the invention: 1,6-diguanidinohexane, 1,9-diaminooxy-nonane, 1,8-diaminooxy-octane, 1,8-diaminooctane, and 1,8-diguanidinooctane, the PQ survival experiment was performed. The compounds were each tested at two concentrations: 0.0625 mM and 0.25 mM. Notably, at these concentrations, the compounds of the invention significantly improved the survival of worms in PQ (for example see FIG. 7). As depicted in FIG. 13, 1,6-Diguanidinohexane significantly improve the survival of worms at both concentrations (p=0.0007 and p<0.0001, respectively). FIG. 14 shows the effect of 1,9-diaminooxy-nonane on worms' survival in PQ (200 mM) at indicated time points. As clearly seen, the compound of the invention significantly improved the survival of worms in PQ. FIG. 15 shows the effect of 1,8-diaminooxy-octane on worms' survival in PQ (200 mM) at indicated time points. As clearly seen, the compound of the invention significantly improved the survival of worms in PQ. FIG. 16 shows the effect of 1,8-diguanidinooctane on worms' survival in PQ (200 mM) at indicated time points. As clearly seen, the compound of the invention significantly improved the survival of worms in PQ.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method of treating or prevention of a disease, disorder, symptom, which is caused by, associated with, or aggravated by impaired mitophagy said method comprising administering a compound having a general formula (I)
R₁-L-R₂ (I)
wherein R₁ and R₂ are each independently selected from —C(═NR₃)NR₄R₅, —NR₆R₇, —N⁺R₈R₉R₁₀, —NR₁₁C(═N)NR₁₂R₁₃, —NR₁₈NR₁₉R₂₀, —ONR₂₂R₂₃, —NR₁₄C(═N)—NR₁₅—C(═N)—NR₁₆R₁₇═N—R₂₁ 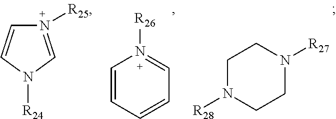
wherein each of R₃-R₂₈ is independently selected from H, straight or branched C₁-C₁₂ alkyl, straight or branched C₂-C₁₂ alkenyl, straight or branched C₂-C₁₂ alkynyl, phenyl, —OH, halogen and any combinations thereof;
L is selected from straight or branched C₆-C₁₂ alkylene, straight or branched C₆-C₁₂ alkenylene, straight or branched C₆-C₁₂ alkynylene;
each defined L is optionally interrupted by at least one of C₄-C₈ cycloalkylene, C₄-C₈ cycloalkenylene, C₄-C₈ cycloalkynylene, arylene, heteroarylene, heteroatom and any combinations thereof;
each defined L is optionally substituted with at least one of halogen and any combinations thereof.

2. A method according to claim 1, wherein L is straight or branched C₆-C₁₂ alkylene.

3. A method according to claim 1, wherein L is interrupted by at least one of C₄-C₈ cycloalkylene, C₄-C₈ cycloalkenylene, C₄-C₈ cycloalkynylene, aryl, heteroaryl, heteroatom and any combinations thereof.

4.-12. (canceled)

13. A method according to claim 1, wherein R₁ and R₂ are each —C(═NR₃)NR₄R₅.

14. A method according to claim 1, wherein R₁ and R₂ are each selected from —NR₆R₇ and —N⁺R₈R₉R₁₀.

15. A method according to claim 1, wherein R₁ and R₂ are each selected from —NR₁₁C(═N)NR₁₂R₁₃ and —NR₁₄C(═N)—NR₁₅—C(═N)—NR₁₆R₁₇.

16. A method according to claim 1, wherein R₁ and R₂ are each —NR₁₈NR₁₉R₂₀.

17. A method according to claim 1, wherein R₁ and R₂ are each ═N—R₂₁.

18. A method according to claim 1, wherein R₁ and R₂ are each —ONR₂₂R₂₃.

19. A method according to claim 1, wherein R₁ and R₂ are each

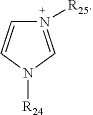

20. A method according to claim 1, wherein R₁ and R₂ are each

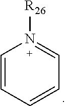

21. A method according to claim 1, wherein R₁ and R₂ are each

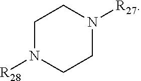

22. (canceled)

23. (canceled)

24. A method according to claim 1, wherein said impaired mitophagy is in non-regenerative tissue.

25. (canceled)

26. A method according to claim 1, wherein said disease, disorder, symptom, which is caused by, associated with, or aggravated by impaired mitophagy is a neurodegenerative disease, disorder and condition associated therewith.

27. A method according to claim 1, wherein said disease, disorder, symptom, which is caused by, associated with, or aggravated by impaired mitophagy is an age-related disease, disorder and condition associated therewith.

28. A method according to claim 1, wherein said disease, disorder, symptom, which is caused by, associated with, or aggravated by impaired mitophagy is selected from Parkinson's disease, Alzheimer's disease, dementia, congestive heart failure, sarcopenia, type 2 diabetes, age-related macular degeneration (AMD), atherosclerosis, cardiovascular diseases, cancer, liver diseases, pancreatic diseases, ocular diseases, arthritis, cataracts, osteoporosis, hypertension, and any combinations thereof.

29. (canceled)

30. A compound having a general formula (I);
R₁-L-R₂ (I)
wherein R₁ and R₂ are each independently selected from —C(═NR₃)NR₄R₅, —NR₆R₇, N⁺R₈R₉R₁₀, —NR₁₁C(═N)NR₁₂R₁₃, —NR₁₄C(═N)—NR₁₅—C(═N)—NR₁₆R₁₇, —NR₁₈NR₁₉R₂₀, ═N—R₂₁, —ONR₂₂R₂₃, 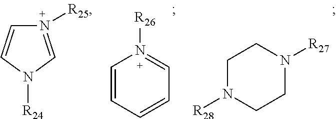
wherein each of R₃-R₂₈ is independently selected from H, straight or branched C₁-C₁₂ alkyl, straight or branched C₂-C₁₂ alkenyl, straight or branched C₂-C₁₂ alkynyl, phenyl, —OH, halogen and any combinations thereof;
L is selected from straight or branched C₆-C₁₂ alkylene, straight or branched C₆-C₁₂ alkenylene, straight or branched C₆-C₁ alkynylene;
each defined L is interrupted by at least one of C₄-C₈ cycloalkylene, C₄-C₈ cycloalkenylene, C₄-C₈ cycloalkynylene, arylene, heteroarylene, heteroatom and any combinations thereof;
each defined L is optionally substituted with at least one of halogen and any combinations thereof.

31.-60. (canceled)
